(19) United States
(12) Patent Application Publication
(10) Pub. No.: US 2004/0171927 A1
(43) Pub. Date: Sep. 2, 2004

(54) Method and apparatus for measuring and compensating for subject motion during scanning

(76) Inventors: Steven Lowen, Burlington, MA (US); Carl Anderson, West Roxbury, MA (US); Michael L. Rohan, Cambridge, MA (US); Perry Renshaw, Bedford, MA (US)

Correspondence Address: CHARLES H. SANDERS, Fish & Richardson P.C., 225 Franklin Street, Boston, MA, 02110-2804, US

(21) Appl. No.: 10/646,858

(22) Filed: Aug. 22, 2003

Related U.S. Application Data
(60) Provisional application No. 60/406,364, filed on Aug. 26, 2002.

(51) Int. Cl. A61B 5/05

(52) U.S. Cl. 600/410

(57) ABSTRACT

This invention provides independent measurement of and dynamic correction for subject movement while a scanning protocol is performed by updating the scanning protocol to compensate for this movement. This simple, easy-to-use, and cost-effective approach offers greater accuracy than previous methods. Using devices with cameras and integrated radiation sources, the invention is suitable for monitoring subject movement by detecting the reflection of the radiation from markers on the subject in real-time and performing dynamic correction for this movement.

### CROSS-REFERENCE TO RELATED APPLICATIONS

This application claims priority to U.S. Provisional Patent Application Serial No. 60/406,364, filed on Aug. 26, 2002, which is incorporated by reference herein in its entirety.

### TECHNICAL FIELD

This invention relates to measurement of and correction for motion during application of scanning technologies, and more particularly to measurement of and correction for motion during magnetic resonance imaging (MRI).

### BACKGROUND

Over 1500 fMRI centers exist worldwide, providing a variety of services to millions of patients every year. A limiting factor in the quality of the images produced is subject motion. In fact, subject motion has been widely recognized as a source of artifacts in MRI data; in 1996, the Institute of Medicine issued a report that identified subject motion as a priority area for future investigation, see IOM: The Committee on the Mathematics and Physics of Emerging Dynamic Biomedical Imaging, the National Research Council, and the Institute of Medicine, Mathematics and Physics of Emerging Biomedical Imaging (Washington, D.C., National Academy Press), 1996. Other scanning technologies, including positron emission tomography (PET) and computerized axial tomography (CAT), suffer similar problems.

In the context of substance abuse research, subject movement is a particularly important concern because, where stimulants are administered, this leads to increased motor activity. In addition, drug-dependent individuals may have co-morbid attention deficit hyperactivity disorders, may develop akathesia during early drug withdrawal, and may have drug-related cognitive impairments associated with increased movement.

Several approaches have been used to address the problem of subject movement. One approach to dealing with subject movement has been to use restraint mechanisms to limit motion during scanning. This has included the use of custom-formed pillows, face masks, bite bars, and stereotactic frames. These restraints are variably tolerated by subjects and do not eliminate residual motion effects. Another approach has been post-processing acquired images to attempt to correct for motion during the acquisition of the images. Techniques such as landmark and surface matching methods have been used in post-processing. Other approaches have been used which do not require operator intervention, including various methods that minimize differences between images, cross-correlation methods, Fourier-based methods, and center of mass tracking.

Several techniques seek to extract information about motion from the data acquired during scanning, without taking action to avoid the effects of motion or actively measure it. One method of seeking to avoid the effects of motion includes synchronizing scanning with a particular point in the breathing cycle, particularly for chest scans. To actively measure movement, navigator echoes, brief scans which can more readily reveal motion effects, are used in conjunction with the main scan protocol in MRI.

### SUMMARY

The present invention achieves the result of independent measurement of and dynamic correction for subject movement by using cameras to detect the reflection of radiation from markers on the subject while the scanning protocol is performed and updating the scanning protocol to compensate for this movement. This simple, easy-to-use, and cost-effective approach offers greater accuracy than previous methods without requiring a complicated, fragile or unwieldy optical set-up or placing a subject at risk. With only devices having cameras and integrated sources of pulsed infrared radiation, the present invention permits monitoring subject movement in real-time and enables dynamic correction for this movement in the scanning protocol while it continues to be performed. Furthermore, by using an external camera system, this approach does not require use of the scanner's resources to probe motion or distort the image collection process and can measure motion simultaneously with, and independently of, the scanning of the subject.

In one aspect of the invention, it features a system having a scanner that performs a scanning protocol on a subject, two or more devices having cameras and integrated sources of radiation that transmit radiation incident on three or more markers on the subject and detect radiation reflected from the markers, and a processor that processes data based on the radiation detected by the cameras and communicates with the scanner to update the scanning protocol to compensate for movement of the subject.

Embodiments of the invention include one or more of the following features. The scanner can be a scanner for any radiative scanning protocol for scanning a subject (e.g., a human head) including, e.g., PET, CAT and MRI. Where the cameras are used with an MR scanner, the cameras experience a magnetic field that decreases in strength with distance from the scanner. One embodiment of the invention includes cameras that can function in a region have a magnetic field strength of more than 100 Gauss, e.g., 900 Gauss, without an appreciable loss of accuracy.

In certain embodiments, the cameras are infrared cameras and the sources of radiation are light-emitting diodes that emit diffuse pulsed infrared radiation. An advantage of these embodiments is that using light-emitting diodes that emit diffuse pulsed infrared radiation does not pose a significant hazard, i.e., risk of blinding, where the subject is a human head. The system can also be adapted to receive any other wavelength of radiation including, e.g., visible, ultraviolet, or fluorescent light. The markers can have a number of different shapes such as approximately hemispherical, spherical, or flat. Reflective hollow plastic spheres make lightweight, robust, inexpensive, and easy-to-implement markers. More than three spheres, e.g., between four and eleven markers, can be easily attached to a typical subject, i.e., a human head, to improve accuracy without causing strain due to weight, adding noticeable cost, or occupying much additional space. Furthermore, defects in the spherical shape due to, e.g., collisions with surfaces, can be tolerated well by the system.

In addition, while in a particular embodiment two cameras are used for simplicity, more cameras—or more markers—can be added for improved resolution. These cameras are located at various angles to the subject; greater angles provide enhanced spatial resolution in the motion parameters. The system can further include mirrors that reflect the radiation incident on the cameras, which allows the cameras to be located at greater angles to the subject. Where two cameras are used, the angle between them and the subject can be, e.g., between 30 and 60 degrees, for instance, approximately 45 degrees. Cameras having accuracy to within 0.1 millimeter or less can be used to provide accurate measurements.

A computer can be used to process the data based on the radiation detected by the cameras and communicate with the scanner to update the scanning protocol to compensate for movement of the subject. This movement can be a translation, rotation, or a combination of a rotation and a translation of the subject. Furthermore, the system can include a display for showing an image of the subject.

In another aspect, the invention features a method of compensating for movement of a subject during scanning that involves performing a scanning protocol on a subject, detecting radiation reflected by three or more approximately spherical markers on the subject, and processing data based on the radiation detected by the cameras and updating the scanning protocol to compensate for motion of the subject. This method can be used, e.g., to diagnose a condition of the subject or test motion correction algorithms.

A further aspect of the invention features a system for updating a scanning protocol performed by a scanner on a subject to compensate for movement by a subject using two or more devices having cameras and integrated sources of radiation that transmit radiation incident on three or more markers on a subject and detect radiation reflected by the markers, and a computer-readable medium having a program that is used by a processor to processes data based on the radiation detected by the cameras and communicate with the scanner to update the scanning protocol to compensate for motion of the subject. In specific embodiments, the computer-readable readable medium is an optical or magnetic storage medium.

Unless otherwise defined, all technical and scientific terms used herein have the same meaning as commonly understood by one of ordinary skill in the art to which the invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a top view of a system of the present invention.

FIGS. 2A and 2B are two graphs of position data from a camera outside a MR scanner.

FIG. 3 is a block diagram of a system of the present invention.

FIG. 4 is a graph of motion data for rotation about the x-axis obtained with a camera system outside a MR scanner compared to a method of estimating motion from data obtained by the MR scanner, statistical parametric mapping (SPM).

FIG. 5 is a graph of motion data for translation along the x-axis obtained with a camera system outside a MR scanner compared to two methods of estimating motion from data obtained by the MR scanner, SPM and decoupled automated rotational and translation (DART).

FIG. 6 is a graph of motion data for rotation about the y-axis obtained with a camera system outside a MR scanner compared to a method of estimating motion from data obtained by the MR scanner, SPM.

FIG. 7 is a graph of motion data for translation along the y-axis obtained with a camera system outside a MR scanner compared to two methods of estimating motion from data obtained by the MR scanner, SPM and DART.

FIG. 8 is a graph of motion data for rotation about the z-axis obtained with a camera system outside a MR scanner compared to two methods of estimating motion from data obtained by the MR scanner, SPM and DART.

FIG. 9 is a graph of motion data for translation along the z-axis obtained with a camera system outside a MR scanner compared to a method of estimating motion from data obtained by the MR scanner, SPM.

Like reference symbols in the various drawings indicate like elements.

### DETAILED DESCRIPTION

The invention features systems, apparatuses, and methods for measuring subject movement, independent of a scanning protocol, by using cameras and dynamically updating the scanning protocol to correct for this movement. The cameras detect subject movement in real-time from radiation emitted by integrated radiation sources that is reflected by three or more markers, permitting dynamic correction for this movement during scanning with an external radiation source, such as an MR scanner.

System

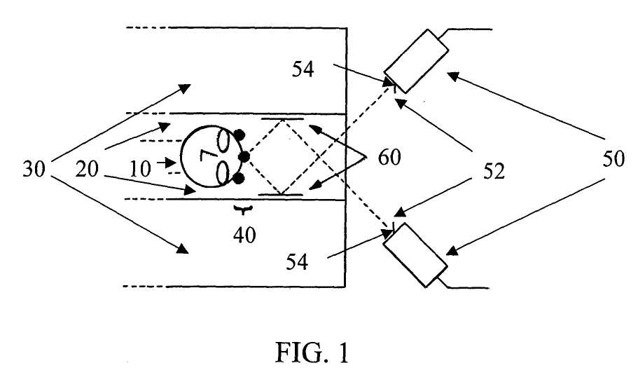

FIG. 1 is a top view of a system of the present invention. The subject 10 is a human head located within the bore 20 of an MR scanner 30. Other objects such as other regions of the body or MR phantoms can also be scanned. There are at least three markers 40 attached to the subject 10. Radiation from the sources of radiation 52 in the devices 50 is reflected by these markers 40 and detected by the cameras 54 in the devices 50 using a system of mirrors 60. The mirrors are an optional feature, which can be used depending on the size of the bore, the space constraints of the external environment of the MR scanner, and the magnetic-field tolerance of the cameras. Using mirrors enables the cameras 54 to be located farther away from the MR scanner, which reduces the magnetic field in the region of the cameras. In addition, the increased angle between the cameras 54 afforded by using mirrors can provide greater accuracy. However, directly measuring the reflected radiation without mirrors simplifies implementation of the system.

In addition to using this system with MRI, it also has application in other scanning technologies, including, e.g., PET and CAT. The system can be implemented with devices having infrared cameras and integrated light-emitting diodes that emit diffuse pulsed infrared radiation (ProReflex system, Qualisys, Inc., East Windsor, Conn.) to detect reflected infrared radiation from markers (Qualisys, Inc., East Windsor, Conn.) mounted on a flexible piece of plastic attached to the top of the subject's head. Where used in conjunction with an MR scanner, the cameras will be located within a magnetic field environment and need to be able to function effectively in this environment. If located close to the MR scanner, they can experience a field strength of, e.g., about 900 Gauss while, if located farther away, the field will be lower, e.g., about 100 Gauss. With other scanning protocols using different types of radiation, the cameras will need to be able to function in those particular fields.

Data Collection

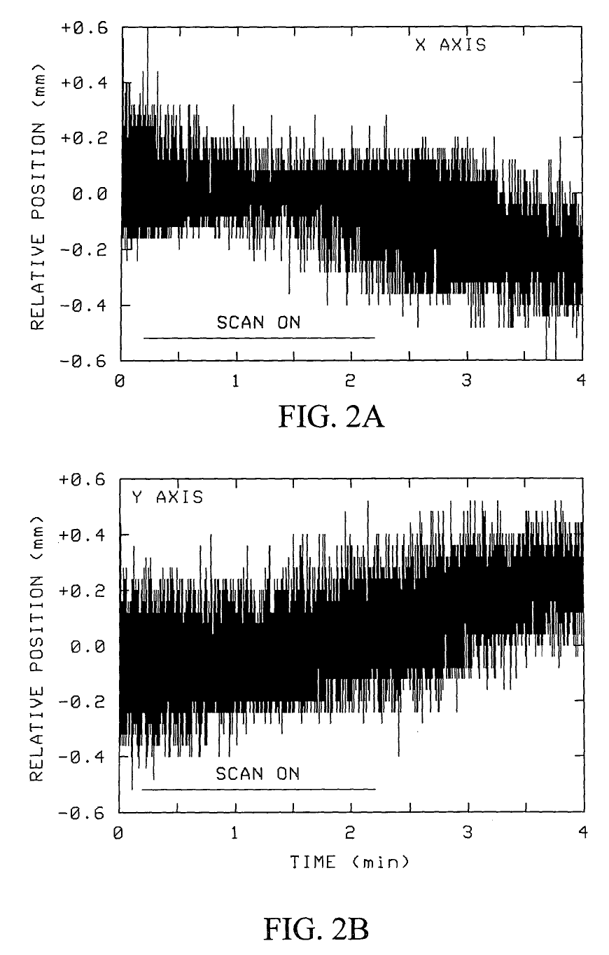

FIGS. 2A and 2B are samples of position data from an infrared camera mounted outside a MR scanner. FIG. 2A shows movement resolved along the x-axis and FIG. 2B shows movement resolved along the y-axis. These graphs demonstrate that a camera can detect subject movement on a submillimeter scale in a magnetic field environment of several hundred Gauss. FIGS. 2A and 2B show that the camera functioned both during the scan and during the time between scans.

Three or more markers will permit spatial resolution along the three axes as well as rotational resolution about these three axes, and a larger number of markers (e.g., five, six, or seven) provides greater accuracy. Since the hollow spherical markers (e.g., from Qualisys, Inc.) are light, inexpensive, and easy-to-implement, accuracy can be improved easily without modification of the camera and integrated light source system. Other geometries of markers, e.g., flat markers, can also be suitable. Used with the ProReflex system, these markers can provide accuracy to within 0.1 millimeter, and other camera systems can offer even greater accuracy. Using more than two cameras also affords greater accuracy. Besides infrared radiation, shorter wavelengths of radiation, e.g., visible or ultraviolet light, can be used instead to reduce the wavelength limit on precision.

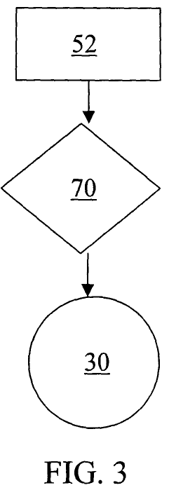

FIG. 3 is a block diagram of a system of the present invention. Information about subject movement collected by the cameras 52 is fed into a processor 70, which dynamically updates the MRI imaging protocol used by the MR scanner 30 to compensate for the subject's motion. The processor can be a computer that is either integrated into or distinct from the MR scanner 30 itself, and the program used by the processor can be obtained from a computer-readable medium, e.g., an optical or magnetic disk. Compensating for the subject's motion during scanning reduces motion artifacts in the images obtained from the scan and facilitates diagnosing whether a subject is experiencing a specified condition, e.g., a neural disorder.

Dynamic Updating of the Scanning Protocol

The motion data obtained from the cameras is used for dynamically updating the MRI scan parameters to compensate for subject movement. By adjusting the slice prescriptions in real time during image collection to reflect updated position information, translating and rotating the image back to its original position during post-processing can be minimized or avoided, and this offers improved image accuracy. A stable image obtained with dynamic updating need not suffer the problems of re-registration and post-collection processing, which are usually employed to account for subject motion. Immediately after each scan, the optical system measures and process as the position information received by the cameras, which takes about eight milliseconds. Next, the new scan position is determined using standard trigonometric methods implemented on a modern scientific workstation which, with proper attention to code optimization, can perform the calculations in much less than a millisecond.

Current and RF pulses are applied to the magnetic coils of an MR scanner perform the desired scanning protocol. In normal operation, the z-coil of an MR system is used for slice prescription and the x and y-coils probe the selected slice. Where the subject moves, the plane of a slice prescription by the z-coil may no longer be parallel to the plane of a pre-movement slice prescription from the subject's perspective. To maintain parallel slicing through the subject from the subject's perspective, a slice prescription can instead be conducted using x and y-coils in conjunction with the gradient coil. The adjustment of the slice prescription is specified by using the standard trigonometric methods to manipulate the data gathered by the cameras. By successively adjusting the slice prescription in this manner, the slice prescriptions can be maintained parallel through the subject from the subject's perspective, which corrects for the subject's motion.

Many MRI scanners implement gradient waveforms in a two-step process. In the first step, scan parameters are calculated in a logical coordinate system, which sets a field of view. In the second, these parameters are mapped to physical coordinates by the application of a rotation matrix in the final stages of the waveform generator. The two steps together take much less than a second, but calculations of position changes can be accomplished within this time and scan position can be rapidly updated by downloading a new rotation matrix to the waveform generator to compensate for subject movement from the previous scan. Thus, the position parameters of the MRI scanning protocol can be updated to compensate for subject motion before the next waveform is generated. Altogether, optical data acquisition, scan position determination, scan parameter calculation, and rotation matrix application comprise less than a second, typically less than the time between scans of the same brain region. Therefore, dynamic updating using position information from a camera system can be applied across a wide variety of MRI scanning protocols. Furthermore, images obtained using dynamic updating of scan protocols to compensate for subject movement can be compared to other images generated using a scanning protocol without dynamic updating after post-processing using motion correction algorithms to identify any algorithms that correlate well.

Example of Motion Detection

Movement was assessed with a ProReflex system from Qualisys, Inc. This system included two devices having infrared cameras with 4 mm lenses, placed 12 feet away from the magnet to prevent significant interactions between the magnetic field and ferrous metals in the cameras. The cameras were also modified to render them less susceptible to the effects of the strong (600 Gauss) magnetic field. The cameras were located at an angle of 30 degrees from the axis of the magnet. These cameras were focused at the top of the patient's head, which was visible through an opening in the bore. Seven approximately spherical, hollow markers (Qualisys, Inc.) were attached to a flexible piece of MRI film and spaced evenly within a circle of radius 5 cm. This assembly was in turn securely attached to the top of the patient's head with collodion and medical tape so that subject movements would be faithfully reflected by the markers' movements. The two cameras acquired independent x-y data from infrared radiation reflected by these markers, from which 3-D coordinates of each marker was obtained in real time. A suitable software package for computing the 3-D coordinates is QTrac (Qualisys, Inc.). The ProReflex camera system provides a resolution of 21.6 arc-seconds, at up to 120 times a second. This translated in real time to 3-D measurements with resolutions of approximately 0.1 mm.

Rotation and translation data were calculated from this 3-D data using standard trigonometric rigid-body algorithms. More complex algorithms can be used to take into account the plasticity of the subject, see, eg., Friston K J et al., “Spatial registration and normalisation of images,” Human Brain Mapping, 2:165-89, 1995. The rotation and translation data obtained using the camera system were compared to results using two methods for estimating motion from MRI data, statistical parametric mapping (SPM) and decoupled automated rotational and translation (DART), see, e.g., Maas, L. C. et al., “Decoupled automated rotational and translational registration for functional MRI time series data: The DART registration algorithm,” Magn. Reson. Imaging, 37:131-139, 1997. SPM was developed for PET image analysis, based on multilinear regression techniques, and has been modified for motion correction of MRI images, see R. Turner et al., “Functional magnetic resonance imaging of the human brain: data acquisition and analysis, Experimental Brain Research, 123:5-12, 1998. DART differs in that it uses rigid body motion correction based on phase comparisons of raw (pre-Fast Fourier transform) MRI data, see L. Mass et al., “Decoupled automated rotational and translational registration for function MRI time series data: the DART registration algorithm,” Magnetic Resonance in Medicine, 37(1):131-39, 1997.

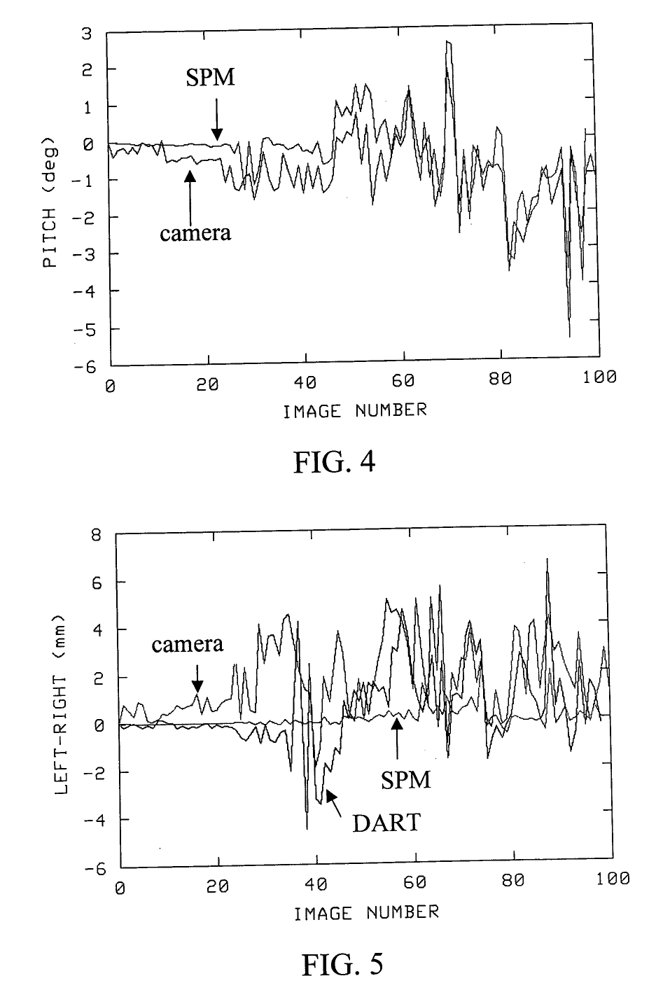

FIG. 4 is a graph of motion data for rotation about the x-axis obtained with a camera system outside an MR scanner compared to an estimate of motion from MRI data obtained using SPM. The x-axis is the axis running left-to-right across a patient lying on his back within the scanner. The rotation about this axis is pitch, and an example of a rotation about this axis is a nod for “yes.” The graph shows relatively good agreement between the motion data obtained directly from the infrared radiation detected by the cameras and the estimated motion data from the SPM algorithm performed on the MRI data. The correlation coefficient between the two lines is 0.801.

FIG. 5 is a graph of motion data for translation along the x-axis compared to estimates of motion from MRI data obtained using SPM and DART. Neither SPM nor DART provide particularly good estimates of the motion data collected directly by the cameras, though the correlation coefficient for DART (0.451) is considerably better than for SPM (0.215).

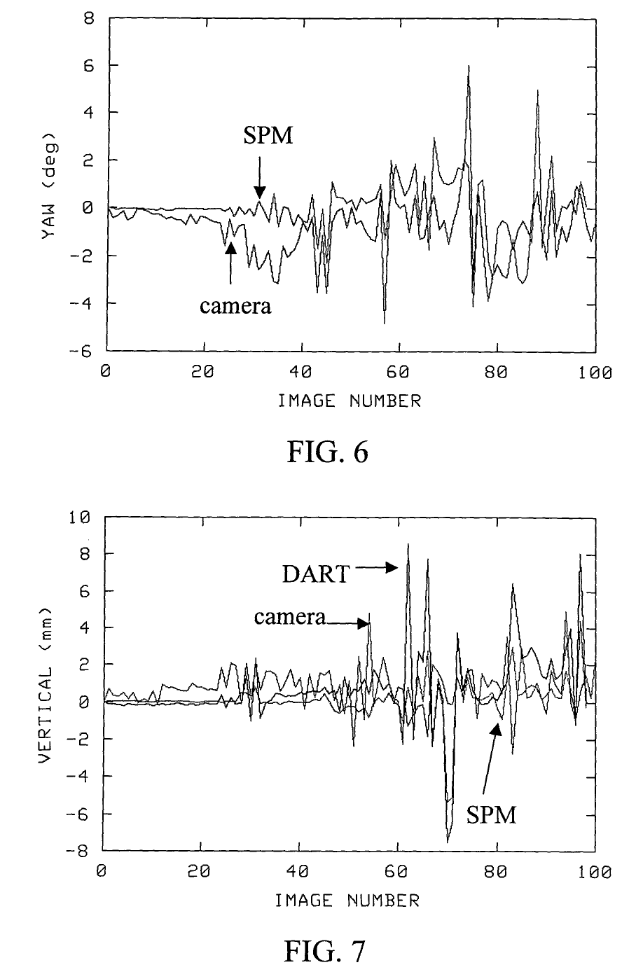

FIG. 6 is a graph of motion data for rotation about the y-axis obtained with a camera system outside a MR scanner compared to an estimate of motion from MRI data obtained using SPM. The y-axis runs perpendicular to the plane of a patient lying in a MR scanner from top to bottom. The rotation about this axis is yaw; an example of a rotation about this axis is a rock of the head from shoulder to shoulder. The motion data from the cameras and the estimate using SPM show fair agreement with a correlation coefficient of 0.622.

FIG. 7 is a graph of motion data for translation along the y-axis compared to estimates of motion from MRI data obtained using SPM and DART. Neither SPM nor DART effectively estimate the motion data collected directly by the cameras, with respective correlation coefficients of 0.008 and 0.115.

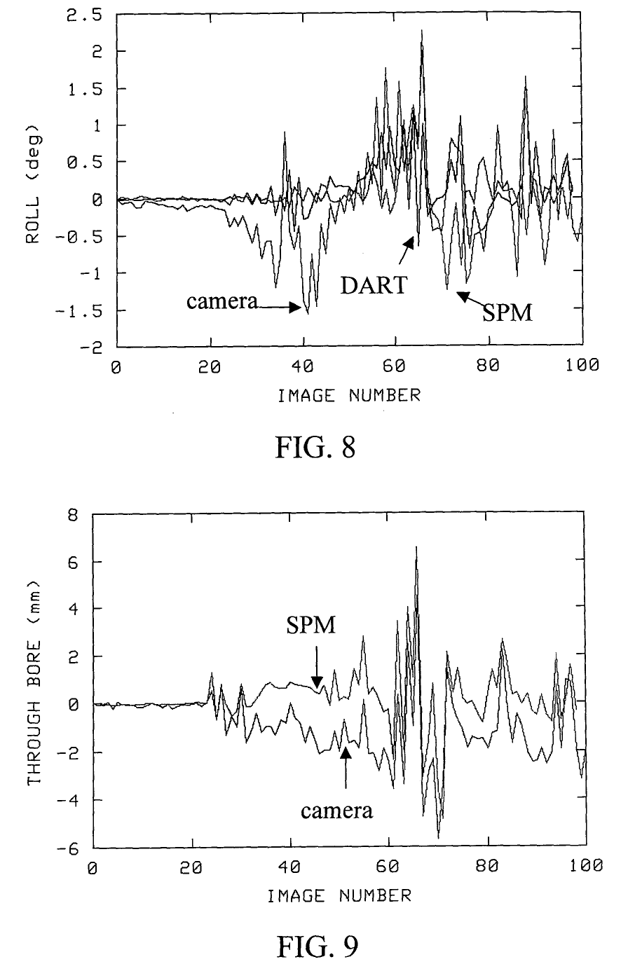

FIG. 8 is a graph of motion data for rotation about the z-axis obtained with a camera system outside a MR scanner compared to an estimate of motion from MRI data obtained using SPM and DART. The z-axis runs parallel to the axis of the bore of the MR scanner. The rotation about this axis is roll; an example of a rotation about this axis is a shake of the head for “no.” While the estimate obtained using DART shows fair agreement with the motion data collected by the camera with a correlation coefficient of 0.547, the estimate obtained using SPM shows little agreement, having a correlation coefficient of 0.266.

FIG. 9 is a graph of motion data for translation along the z-axis compared to an estimate of motion from MRI data obtained using SPM. Using SPM provides a good estimate of the motion data collected directly by the cameras, with a correlation coefficient of 0.729.

### Other Embodiments

A number of embodiments of the invention have been described. Nevertheless, it will be understood that various modifications may be made without departing from the spirit and scope of the invention. For example, the invention can be adapted for application in PET and CAT. Accordingly, other embodiments are within the scope of the following claims.

What is claimed is:

1. A system comprising:
(a) a scanner that performs a scanning protocol on a subject;
(b) two or more devices each comprising cameras and integrated sources of radiation that transmit radiation incident on three or more markers on the subject and detect the radiation reflected by the markers; and
(c) a processor that processes data based on the radiation detected by the cameras and communicates with the scanner to update the scanning protocol to compensate for movement of the subject.

2. The system of claim 1, wherein the scanner is a magnetic resonance scanner.

3. The system of claim 2, wherein the cameras can function in a region having a magnetic field strength of more than 100 Gauss without an appreciable loss of accuracy.

4. The system of claim 1, wherein the cameras are infrared cameras and the sources of radiation are light-emitting diodes that emit diffuse pulsed infrared radiation.

5. The system of claim 1, wherein two cameras detect the radiation.

6. The system of claim 5, wherein the angle formed between the two devices and the axis of the scanner is approximately 45 degrees.

7. The system of claim 5, wherein the angle formed between the two devices and the axis of the scanner is between approximately 30 and 60 degrees.

8. The system of claim 1, wherein the cameras are accurate to within 0.1 millimeter or less.

9. The system of claim 1, wherein the processor is a computer.

10. The system of claim 1, further comprising two or more mirrors that reflect radiation to be incident on the cameras.

11. The system of claim 1, wherein the markers are approximately spherical.

12. The system of claim 1, wherein the number of markers is more than three.

13. The system of claim 1, further comprising:
(d) a display that displays an image of the subject.

14. A method of compensating for movement of a subject during scanning, the method comprising:
(a) performing a scanning protocol on a subject;
(b) detecting diffuse radiation reflected by three or more approximately spherical markers on the subject; and
(c) processing data based on the radiation detected by the cameras and updating the scanning protocol to compensate for motion of the subject.

15. The method of claim 14, wherein the scanning protocol is a magnetic resonance imaging protocol.

16. The method of claim 14, wherein the scanning protocol is a positron emission tomography protocol or a computer axial tomography protocol.

17. The method of claim 14, wherein the radiation is pulsed infrared radiation.

18. The method of claim 14, wherein the number of markers is more than three.

19. The method of claim 14, further comprising diagnosing a condition of the subject using results from the scanning protocol.

20. The method of claim 14, further comprising testing motion correction algorithms.

21. A system for updating a scanning protocol performed by a scanner on a subject to compensate for movement by the subject, comprising:
(a) two or more devices each comprising cameras and integrated sources of radiation that transmit radiation incident on three or more markers on the subject and detect radiation reflected by the markers; and
(b) a computer-readable medium having a program that is used by a processor to processes data based on the radiation detected by the cameras and communicate with the scanner to update the scanning protocol to compensate for motion of the subject.

22. The system of claim 21, wherein the cameras are infrared cameras and the sources of radiation are light-emitting diodes that emit diffuse pulsed infrared radiation.

23. The system of claim 21, wherein two cameras detect the radiation.

24. The system of claim 21, wherein the cameras can function in a region having a magnetic field strength of more than 100 Gauss without an appreciable loss of accuracy.

25. The system of claim 21, wherein the computer-readable medium is an optical or magnetic storage medium.

26. The system of claim 21, wherein the scanning protocol is a magnetic resonance imaging protocol.

27. A magnetic resonance system for updating a magnetic resonance imaging protocol to compensate for movement of a subject, comprising:
(a) a magnetic resonance scanner that performs the magnetic resonance imaging protocol on the subject;
(b) two cameras that detect pulsed infrared radiation emitted by light-emitting diodes that is reflected by at least three spherical markers on the subject; and
(c) a processor that processes data based on the infrared radiation detected by the cameras and communicates with the magnetic resonance scanner to update the magnetic resonance imaging protocol to compensate for movement of the subject.

28. The magnetic resonance system of claim 27, wherein the movement is a rotation, a translation, or a combination of a rotation and a translation of the subject.

29. The magnetic resonance system of claim 27, wherein the subject is a human head.
